(19) United States
(12) Patent Application Publication
(10) Pub. No.: US 2014/0121598 A1
(43) Pub. Date: May 1, 2014

(54) LIQUID TRANSPORT APPARATUS AND CATHETER COMING-OFF DETERMINATION METHOD

(71) Applicant: Seiko Epson Corporation, Tokyo (JP)

(72) Inventor: Makoto KATASE, Azumino-shi (JP)

(73) Assignee: Seiko Epson Corporation, Tokyo (JP)

(21) Appl. No.: 14/063,447

(22) Filed: Oct. 25, 2013

(30) Foreign Application Priority Data
Oct. 30, 2012 (JP) 2012-238673

(51) Int. Cl. A61M 5/168; A61M 5/142

(52) U.S. Cl. CPC A61M 5/16831; A61M 5/14244
USPC 604/111

(57) ABSTRACT

A liquid transport apparatus includes a tube for constituting a flow channel transporting a liquid, plural fingers that push and block the tube, a cam that pushes the fingers in sequence so as to squeeze the tube to transport the liquid, a catheter for injecting the liquid into a living body, a first electrode that is provided further toward the downstream side than a region pushed by the plural fingers and is made to come into contact with the liquid, a second electrode made to come into contact with the living body, and a determination unit that determines that the catheter comes off the living body on the basis of the impedance between the first electrode and the second electrode.

### BACKGROUND

1. Technical Field

The present invention relates to a liquid transport apparatus and a catheter coming-off determination method.

2. Related Art

As a liquid transport apparatus that transports a liquid, a micro pump described in JP-A-2010-77947 is known. In the micro pump, plural fingers are arranged along a tube, and as a cam sequentially pushes the fingers, the tube is squeezed to transport the liquid.

It is considered that the liquid transported using such a liquid transport apparatus is injected into a living body via a catheter.

### SUMMARY

An advantage of some aspects of the invention is to determine that a catheter has come off a living body.

An aspect of the invention is directed to a liquid transport apparatus including a tube for constituting a flow channel transporting a liquid, plural fingers that push and block the tube, a cam that pushes the fingers in sequence so as to squeeze the tube to transport the liquid, a catheter for injecting the liquid into a living body, a first electrode that is provided further toward the downstream side than a region pushed by the plural fingers and is made to come into contact with the liquid, a second electrode made to come into contact with the living body, and a determination unit that determines that the catheter comes off the living body on the basis of the impedance between the first electrode and the second electrode.

Other features of the invention will become clear from the description of the present specification and the accompanying drawings.

### BRIEF DESCRIPTION OF THE DRAWINGS

The invention will be described with reference to the accompanying drawings, wherein like numbers reference like elements.

FIG. 1 is an overall perspective view of a liquid transport apparatus.

FIG. 2 is an exploded view of the liquid transport apparatus.

FIG. 3 is a cross-sectional view of the liquid transport apparatus.

FIG. 4 is a projected top view of the inside of the liquid transport apparatus.

FIG. 5 is a schematic explanatory view of a pump unit.

FIG. 6 is an exploded perspective view showing an internal configuration of a main body.

FIG. 7 is a perspective view of a rear surface of the main body.

FIG. 8 is an exploded perspective view showing an internal configuration of a cartridge.

FIG. 9 is an exploded perspective view of a rear surface of a base of the cartridge.

FIG. 10 is a perspective view when the liquid transport apparatus is viewed from a bottom surface side of a patch.

FIG. 11 is a flow diagram showing a method of using the liquid transport apparatus.

FIG. 12 is an explanatory view of priming processing.

FIG. 13 is an explanatory view of a monitoring device that monitors coming-off of a soft needle.

### DESCRIPTION OF EXEMPLARY EMBODIMENTS

At least the following matters become evident from the description of the present specification and the accompanying drawings.

A liquid transport apparatus which includes a tube for constituting a flow channel transporting a liquid, plural fingers that push and block the tube, a cam that pushes the fingers in sequence so as to squeeze the tube to transport the liquid, a catheter for injecting the liquid into a living body, a first electrode that is provided further toward the downstream side than a region pushed by the plural fingers and is made to come into contact with the liquid, a second electrode made to come into contact with the living body, and a determination unit that determines that the catheter comes off the living body on the basis of the impedance between the first electrode and the second electrode becomes evident.

According to such a liquid transport apparatus, the coming-off of the catheter can be determined on the basis of the impedance between the first electrode and the second electrode having the liquid and the living body as an electric conduction path.

It is desirable to have a replaceable cartridge and a patch that remains adhered to the living body when the cartridge is replaced, and it is desirable that the cartridge has a connecting needle serving as the first electrode and that the patch has the catheter, the second electrode, and a septum, and the liquid of the cartridge is transported to the catheter via the connecting needle by the connecting needle being inserted through the septum. This enables the first electrode to be arranged close to the living body.

It is desirable that the catheter is made of insulating materials. This can suppress short circuiting.

It is desirable that the second electrode is constituted by a conductive adhesion pad. This can increase the area by which the second electrode comes into contact with the living body.

It is desirable that the adhesion pad is formed with a hole through which the catheter passes. Accordingly, since the catheter does not come off easily and an injection port of the catheter is arranged in the vicinity of the second electrode, the conduction paths going via the liquid and the living body are easily formed.

It is desirable that there is no conductivity at a peripheral portion of the adhesion pad and there is conductivity at a central portion. This reduces an influence exerted on the measurement of the impedance even if the peripheral portion of the adhesion pad becomes unstuck.

When the impedance between the first electrode and the second electrode is measured, it is desirable to apply an alternating voltage to the first electrode and the second electrode so that a bias voltage is not applied between the first electrode and the second electrode. This can keep an electrochemical process from occurring in the liquid.

In a catheter coming-off determination method of a liquid transport apparatus including a tube for constituting a flow channel transporting a liquid, plural fingers that push and block the tube, a cam that pushes the fingers in sequence so as to squeeze the tube to transport the liquid, and a catheter for injecting the liquid into a living body, the catheter coming-off determination method including determining that the catheter comes off the living body on the basis of the impedance between a first electrode that is provided further toward the downstream side than a region pushed by the plural fingers and is made to come into contact with the liquid, and a second electrode made to come into contact with the living body becomes evident. This enables the coming-off of the catheter to be determined.

### EMBODIMENTS

### Basic Configuration of Liquid Transport Apparatus

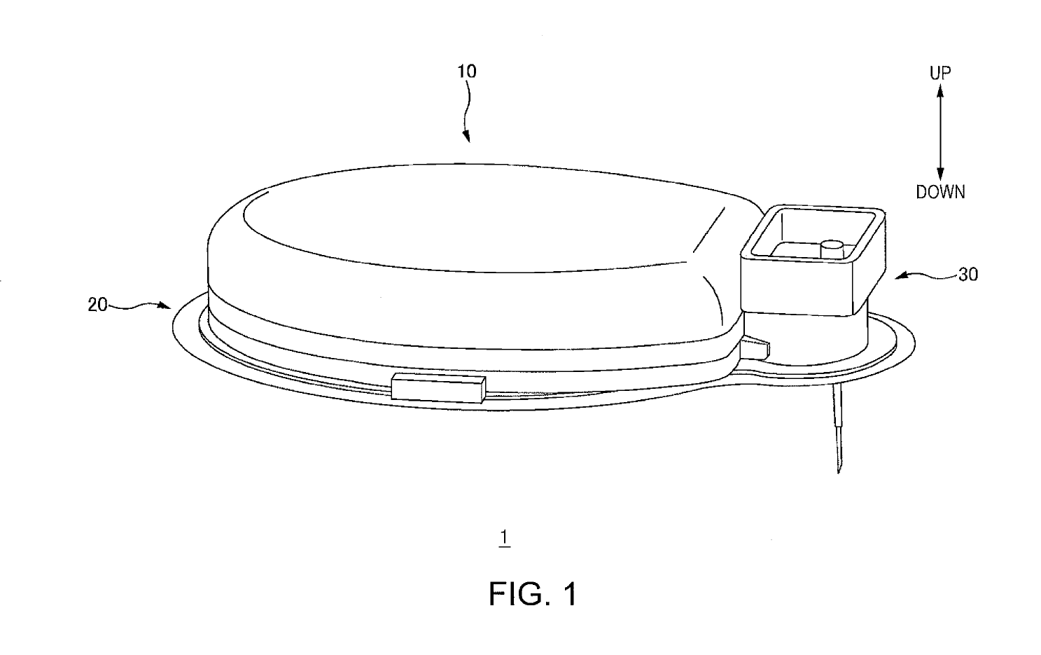

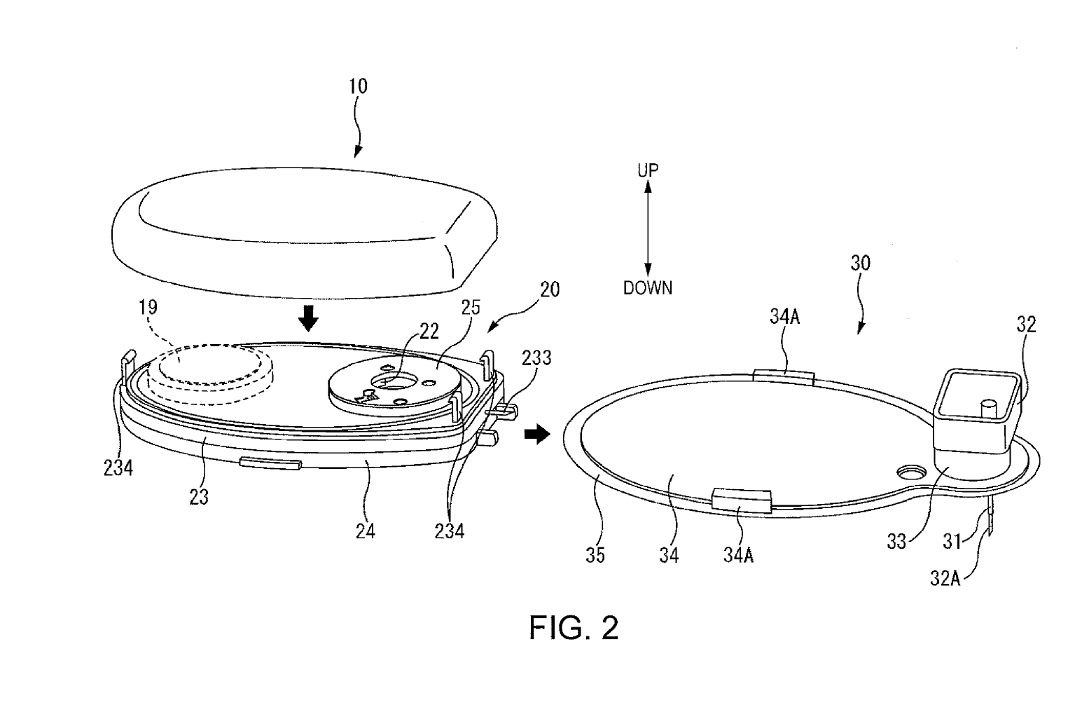

FIG. 1 is an overall perspective view of a liquid transport apparatus 1. FIG. 2 is an exploded view of the liquid transport apparatus 1. As shown in the drawings, description may be made with a side (living body side) to which the liquid transport apparatus 1 is adhered being defined as “down”, and its opposite side being defined as “up”.

The liquid transport apparatus 1 is an apparatus for transporting a liquid. The liquid transport apparatus 1 includes a main body 10, a cartridge 20, and a patch 30. The main body 10, the cartridge 20, and the patch 30 are separable as shown in FIG. 2, but are integrally assembled as shown in FIG. 1 in use. The liquid transport apparatus 1 is favorably used, for example, for adhering the patch 30 to a living body and regularly injecting insulin stored in the cartridge 20. When the liquid (for example, insulin) stored in the cartridge 20 runs out, the cartridge 20 is replaced, but the main body 10 and the patch 30 continue being used. However, the patch 30 is also replaced at low frequency.

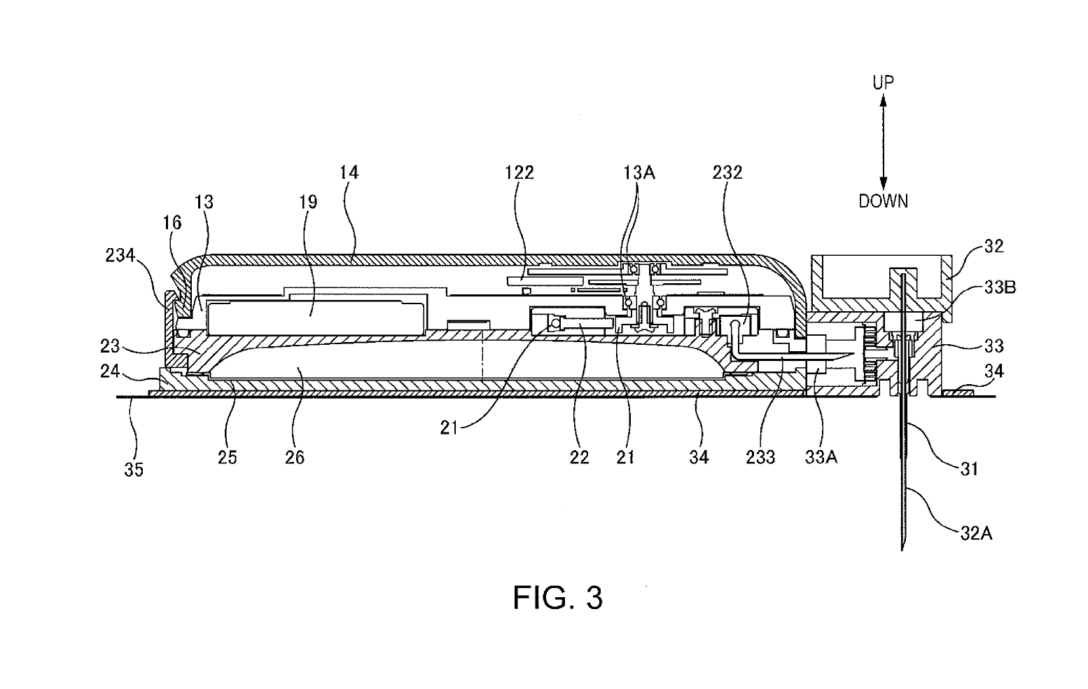

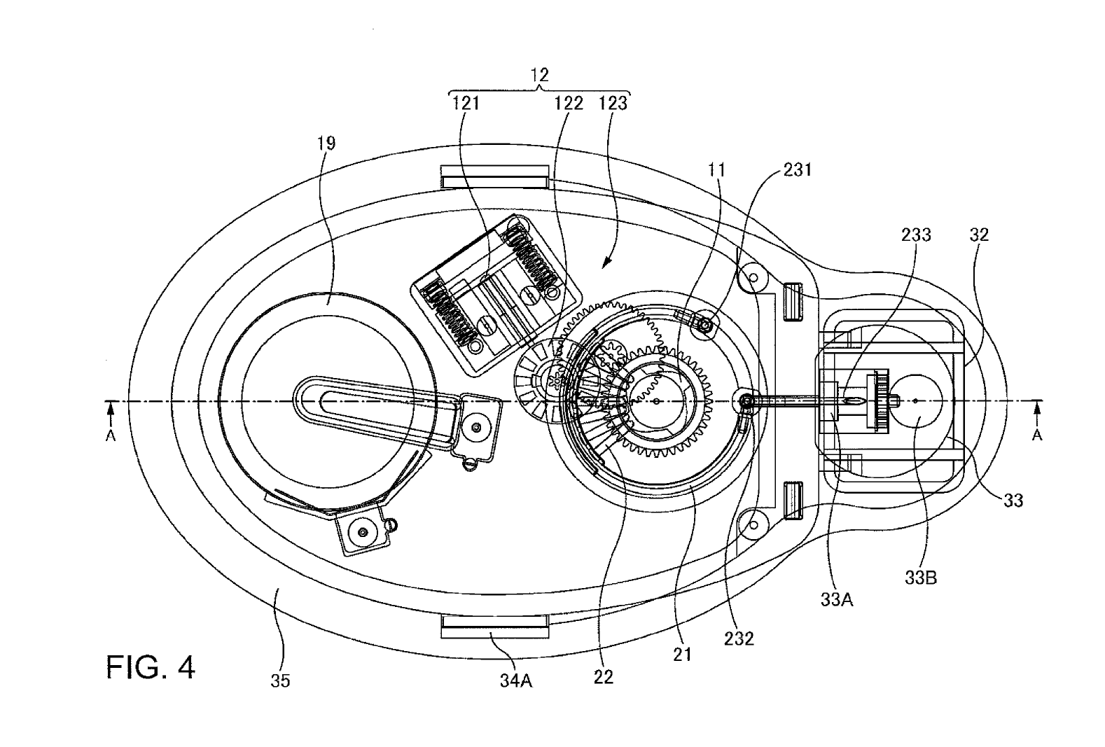

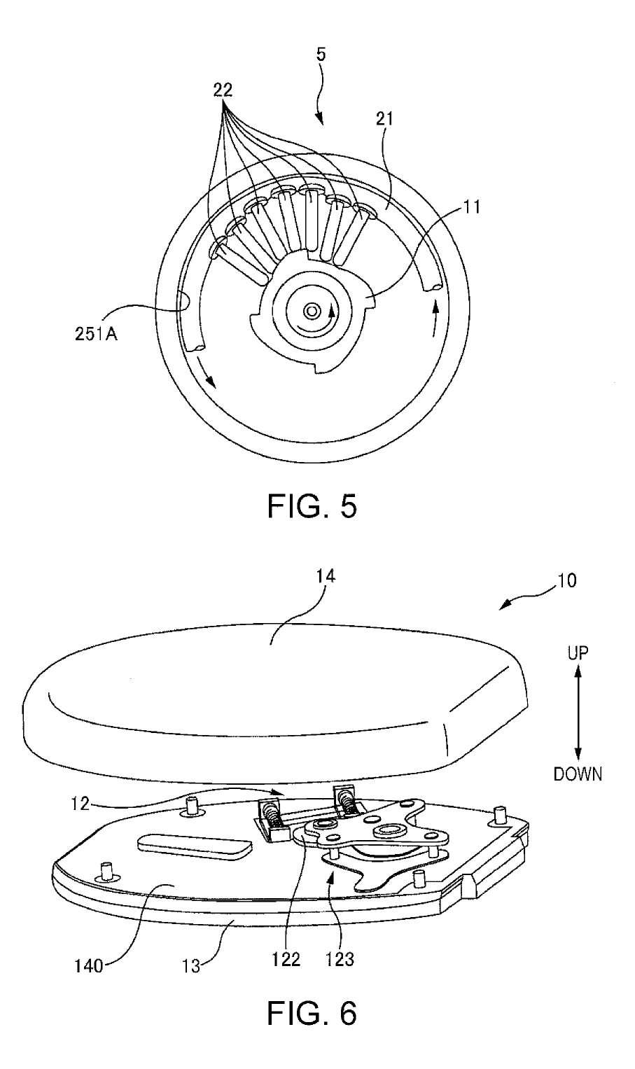

FIG. 3 is a cross-sectional view of the liquid transport apparatus 1. FIG. 4 is a projected top view of the inside of the liquid transport apparatus 1. The configuration of a pump unit 5 is also shown in FIG. 4. FIG. 5 is a schematic explanatory view of the pump unit 5.

The pump unit 5 has a function as a pump for transporting the liquid stored in the cartridge 20, and includes a tube 21, plural fingers 22, a cam 11, and a drive mechanism 12.

The tube 21 is a tube for transporting the liquid. An upstream side (upstream side when being based on a transport direction of the liquid) of the tube 21 communicates with a liquid storage portion 26 of the cartridge 20. The tube 21 has elasticity to such a degree that the tube is blocked when being pushed by the fingers 22 and is returned to its original state if the force from the fingers 22 is released. The tube 21 is arranged partially in the shape of a circular arc shape along an inner surface of a tube guide wall 251A of the cartridge 20. The circular-arc-shaped portion of the tube 21 is arranged between the inner surface of the tube guide wall 251A and the plural fingers 22. The center of the circular arc of the tube 21 coincides with the rotation center of the cam 11.

The fingers 22 are members for blocking the tube 21. The fingers 22 receive a force from the cam 11 and operate in a following manner. The fingers 22 have a rod-shaped shaft portion and a collar-shaped pressing portion, and are formed in a T-shape. The rod-shaped shaft portion comes into contact with the cam 11, and the collar-shaped pressing portion comes into contact with the tube 21. The fingers 22 are supported so as to be movable along the axial direction.

The plural fingers 22 are arranged at equal intervals radially from the rotation center of the cam 11. The plural fingers 22 are arranged between the cam 11 and the tube 21. Here, seven fingers 22 are provided. In the following description, the plural fingers may be referred to as a first finger 22A, a second finger 22B, . . . , and a seventh finger 22G in sequence from the upstream side in the transport direction of the liquid.

The cam 11 has projection portions in four places of an outer periphery thereof. The plural fingers 22 are arranged at the outer periphery of the cam 11, and the tube 21 is arranged outside the fingers 22. The tube 21 is blocked by the fingers 22 being pushed by the projection portions of the cam 11. If the fingers 22 come off the projection portions, the tube 21 returns to its original shape by the elastic force of the tube 21. If the cam 11 rotates, the seven fingers 22 are pushed in sequence from the projection portions, and the tube 21 is blocked sequentially from the upstream side in the transport direction. This causes the tube 21 to perform a peristaltic motion so as to squeeze and transport the liquid. In order to prevent flowback of the liquid, the projection portions of the cam 11 are formed so that at least one or preferably two fingers 22 block the tube 21.

The drive mechanism 12 is a mechanism for rotating the cam 11. The drive mechanism 12 has a piezoelectric motor 121, a rotor 122, and a reduced speed transmission mechanism 123 (refer to FIG. 4).

The piezoelectric motor 121 is a motor for rotating the rotor 122 using vibration of piezoelectric devices. The piezoelectric motor 121 applies a driving signal to the piezoelectric devices bonded on both faces of a rectangular vibrating body, to thereby vibrate the vibrating body. An end portion of the vibrating body comes into contact with the rotor 122, and if the vibrating body vibrates, the end portion vibrates while drawing predetermined tracks, such as an elliptical track or an 8-shaped track. When the end portion of the vibrating body comes into contact with the rotor 122 in a portion of a vibrating track, the rotor 122 is rotationally driven. The piezoelectric motor 121 is biased toward the rotor 122 with a pair of springs so that the end portion of the vibrating body comes into contact with the rotor 122.

The rotor 122 is a driven body rotated by the piezoelectric motor 121. The rotor 122 is formed with a rotor pinion that constitutes a portion of the reduced speed transmission mechanism 123.

The reduced speed transmission mechanism 123 is a mechanism that transmits the rotation of the rotor 122 to the cam 11 in a predetermined reduction ratio. The reduced speed transmission mechanism 123 is constituted by the rotor pinion, a transmission wheel, and a cam gear. The rotor pinion is a pinion integrally attached to the rotor 122. The transmission wheel has a main gear that meshes with the rotor pinion and a pinion that meshes with the cam gear, and has a function to transmit the rotative force of the rotor 122 to the cam 11. The cam gear is integrally attached to the cam 11 and is rotatably supported together with the cam 11.

Among the tube 21, the plural fingers 22, the cam 11, and the drive mechanism 12 that constitute the pump unit 5, the cam 11 and the drive mechanism 12 are provided at the main body 10, and the tube 21 and the plural fingers 22 are provided at the cartridge 20. Hereinafter, the configuration of the main body 10, the cartridge 20, and the patch 30 will be described.

### Main Body 10

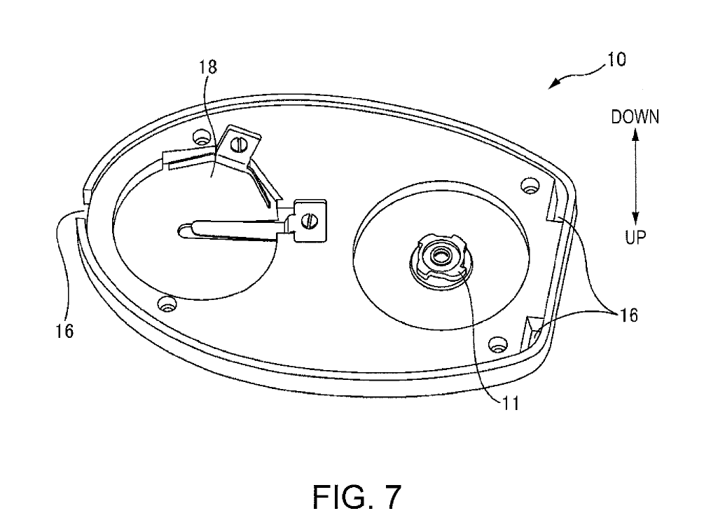

FIG. 6 is an exploded perspective view showing an internal configuration of the main body 10. FIG. 7 is a perspective view of a rear surface of the main body 10. Hereinafter, the configuration of the main body 10 will be described referring to FIGS. 1 to 4 together with these drawings.

The main body 10 has a main body base 13 and a main body case 14. The aforementioned drive mechanism 12, and a control board 15 (control unit) that performs control of the piezoelectric motor 121 or the like are provided on the main body base 13. The drive mechanism 12 (the piezoelectric motor 121, the rotor 122, the reduced speed transmission mechanism 123) and the control board 15 on the main body base 13 are covered with and protected by the main body case 14.

The main body base 13 is provided with a bearing 13A. A rotating shaft of the cam 11 penetrates the main body base 13, and the bearing 13A rotatably supports the rotating shaft of the cam 11 with respect to the main body base 13. The cam 11 is integral with the cam gear that constitutes the reduced speed transmission mechanism 123, and the cam gear is covered with the main body case 14 and is arranged inside the main body 10, and the cam 11 is exposed from the main body 10. If the main body 10 and the cartridge 20 are combined together, the cam 11 exposed from the main body 10 meshes with end portions of the fingers 22 of the cartridge 20.

The main body 10 is provided with a hook catch 16. A fixing hook 234 of the cartridge 20 is caught in the hook catch 16 to fix the main body 10 to the cartridge 20.

Additionally, the main body 10 has a battery housing portion 18. A battery 19 housed in the battery housing portion 18 serves as an electric power source of the liquid transport apparatus 1.

### Cartridge 20

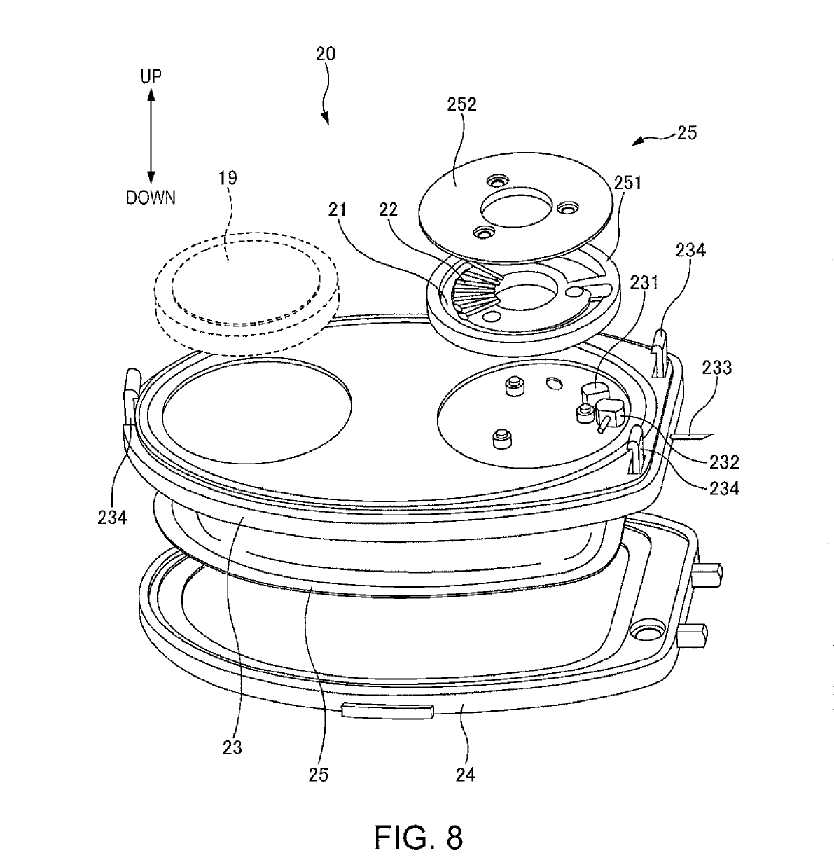

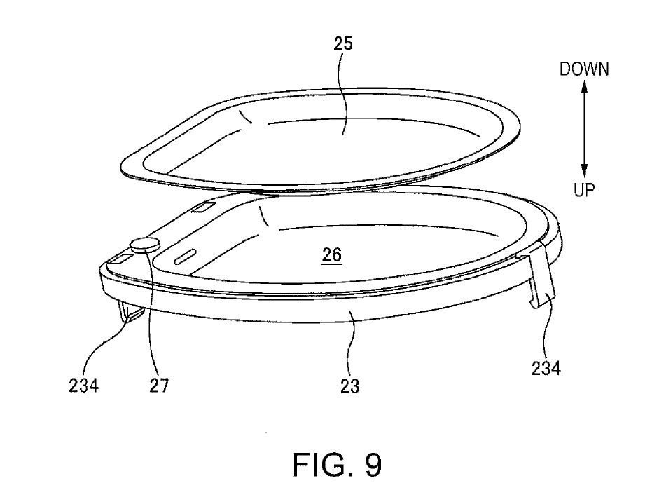

FIG. 8 is an exploded perspective view showing an internal configuration of the cartridge 20. FIG. 9 is an exploded perspective view of a rear surface of a base of the cartridge 20. Hereinafter, the configuration of the cartridge 20 will be described referring to FIGS. 1 to 5 together with these drawings.

The cartridge 20 has a cartridge base 23 and a base receptacle 24.

A tube unit 25 is provided on the upper side of the cartridge base 23. The tube unit 25 has the afore-mentioned tube 21 and plural fingers 22, a unit base 251, and a unit cover 252. The unit base 251 is formed with the tube guide wall 251A, and the tube 21 is arranged in the shape of a circular arc inside the unit base 251. Additionally, the unit base 251 supports the fingers 22 so as to be movable in the axial direction. The tube 21 and the fingers 22 within the unit base 251 are covered with the unit cover 252.

The tube unit 25 is formed in a flat cylindrical shape, and the cam 11 exposed from the main body 10 is inserted into a central cavity of the tube unit 25. This allows the fingers 22 on the cartridge 20 side to mesh with the cam 11 on the main body 10 side.

The cartridge base 23 is provided with a supply-side joint 231 and a discharge-side joint 232. End portions of the tube 21 within the tube unit 25 are connected to the supply-side joint 231 and the discharge-side joint 232, respectively. If the plural fingers 22 squeeze the tube 21 in sequence, the liquid is supplied from the supply-side joint 231 to the tube 21 and the liquid is discharged from the discharge-side joint 232. A connecting needle 233 communicates with the discharge-side joint 232, and the liquid discharged from the discharge-side joint 232 is supplied to the patch 30 side via the connecting needle 233.

The cartridge base 23 is formed with the fixing hook 234. The fixing hook 234 is caught in the hook catch 16 of the main body 10 to fix the main body 10 to the cartridge 20.

A reservoir film 25 is pinched between the cartridge base 23 and the base receptacle 24. The periphery of the reservoir film 25 is tightly bonded to a bottom surface of the cartridge base 23. The storage portion 26 is formed between the cartridge base 23 and the reservoir film 25, and the liquid (for example, insulin) is stored in the storage portion 26. The storage portion 26 communicates with the supply-side joint 231, and the liquid stored in the storage portion 26 is supplied to the tube 21 via the supply-side joint 231.

The storage portion 26 is configured on the lower side of the cartridge base 23 as described above. Since the tube 21 and the fingers 22 that constitute the pump unit 5 are arranged on the upper side of the cartridge base 23, the pump unit 5 and the storage portion 26 are arranged vertically. This achieves miniaturization of the liquid transport apparatus 1. Additionally, the storage portion 26 is arranged further toward the living body side than the pump unit 5. This easily keeps the liquid stored in the storage portion 26 warm at the body temperature of a living body and suppresses the difference between the temperature of the liquid and the body temperature of the living body.

If the liquid stored in the storage portion 26 runs out, the cartridge 20 is removed from the liquid transport apparatus 1 and replaced with a new cartridge 20. However, it is possible to inject the liquid from the outside via a cartridge septum 27 into the storage portion 26 using an injection needle. In addition, the cartridge septum 27 is made of material (for example, rubber, silicon, or the like) that closes a hole if the injection needle is extracted.

### Patch 30

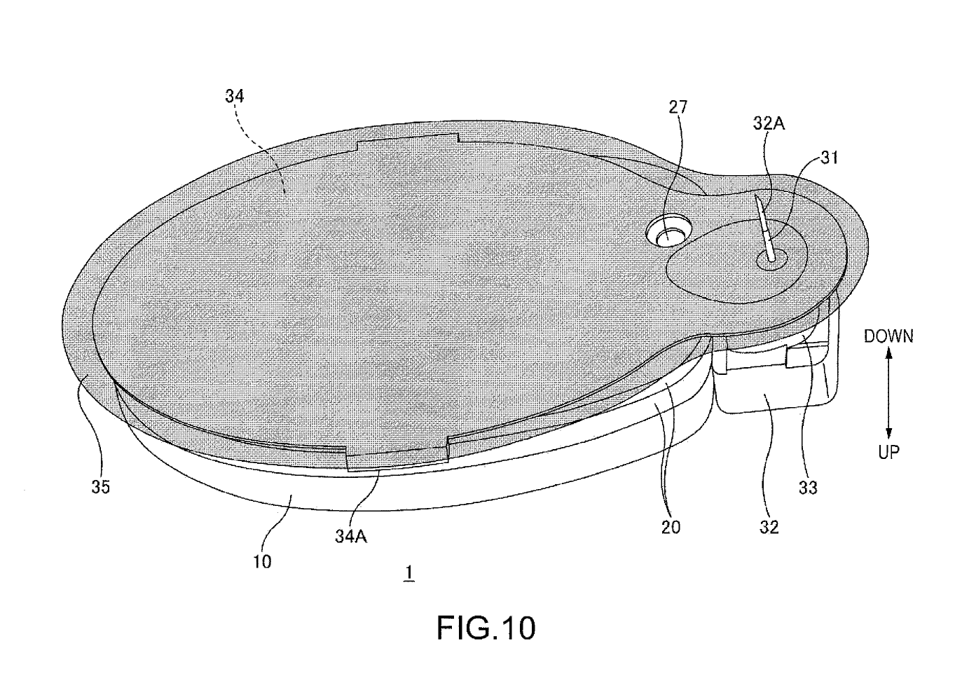

FIG. 10 is a perspective view when the liquid transport apparatus 1 is viewed from the bottom surface side of the patch 30. Hereinafter, the configuration of the patch 30 will be described also referring to FIGS. 1 to 5.

The patch 30 has a soft needle 31, an introduction needle folder 32, a port base 33, a patch base 34, and an adhesion pad 35.

The soft needle 31, which is a tube for injecting the liquid into the living body, functions as a catheter. The soft needle 31 is made of, for example, flexible material, such as fluororesin. One end of the soft needle 31 is fixed to the port base 33.

The introduction needle folder 32 is a member that holds an introduction needle 32A. One end of the introduction needle 32A is fixed to the introduction needle folder 32. The introduction needle 32A is a metal needle for inserting the flexible soft needle 31 into the living body. The introduction needle 32A is an elongated hollow tubular needle, and has a lateral hole (not shown). If the liquid is supplied from the lateral hole of the introduction needle 32A, the liquid is discharged from the tip of the introduction needle 32A. This enables priming processing of causing the inside of the flow channel of the liquid transport apparatus 1 to be filled with the liquid before the soft needle 31 punctures the living body.

In the state before use, the introduction needle folder 32 is attached to the port base 33, and the introduction needle 32A is inserted through the soft needle 31 and a needle point thereof is exposed from the lower side of the soft needle 31. When the patch 30 is pasted on the living body, after the soft needle 31 has punctured the living body together with the introduction needle 32A, the introduction needle folder 32 is pulled out (extracted) from the port base 33 together the introduction needle 32A. Since the hard introduction needle 32A does not continue to be indwelled in the living body, a burden on the living body is small. In addition, although the soft needle 31 continues being indwelled in the living body, since the soft needle 31 is soft, the load to the living body is small.

The port base 33 is a member that supplies the liquid, which is supplied from the connecting needle 233 of the cartridge 20, to the soft needle 31. The port base 33 has a septum 33A for a connecting needle, and a septum 33B for an introduction needle. The septum 33A for a connecting needle and the septum 333 for an introduction needle are made of material (for example, rubber, silicon, or the like) that closes a hole if the needle is extracted. The connecting needle 233 of the cartridge 20 is inserted through the septum 33A for a connecting needle, and the liquid is supplied from the cartridge 20 side to the patch 30 side through the septum 33A for a connecting needle via the connecting needle 233. Even if the connecting needle 233 of the cartridge 20 is extracted from the patch 30 for replacement of the cartridge 20, a hole of the septum 33A for a connecting needle formed by the connecting needle 233 is spontaneously closed. The introduction needle 32A is inserted through the septum 33B for an introduction needle, and if the introduction needle 32A is pulled out, a hole of the septum 33B for an introduction needle formed by the introduction needle 32A is spontaneously closed. The septum 33A for a connecting needle and the septum 33B for an introduction needle prevent the liquid within the patch 30 from leaking to the outside as well as prevent bodily fluids of the living body from flowing back to the patch 30 side. In addition, a region (regions other than the septum for an introduction needle) where the introduction needle 32A is present within the port base 33 serves as a flow channel for the liquid after the extraction of the introduction needle 32A.

The patch base 34 is a plate-shaped member fixed to the port base 33. The patch base 34 has a fixing portion 34A for fixing the base receptacle 24. The adhesion pad 35 is attached to a bottom surface of the patch base 34. The adhesion pad 35 is an adhesive pad for adhering the patch 30 to the living body or the like.

In the above liquid transport apparatus 1, the pump unit 5 and the storage portion 26 are arranged vertically, and the downsizing of the liquid transport apparatus 1 is achieved. This enables the adhesion pad 35 to be downsized.

### Basic Using Method

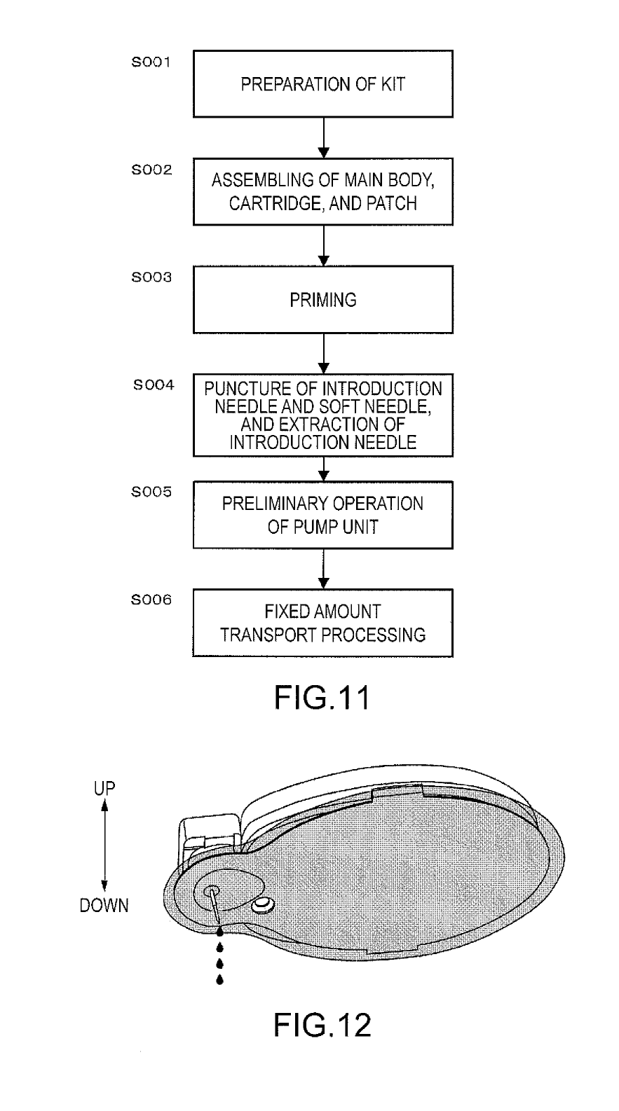

FIG. 11 is a flow diagram showing a method of using the liquid transport apparatus 1.

First, a user prepares a kit that is the liquid transport apparatus 1 (S001). The main body 10, the cartridge 20, and the patch 30 for constituting the liquid transport apparatus 1 are included in the kit. As shown in FIG. 2, the user assembles the main body 10, the cartridge 20, and the patch 30 to assemble the liquid transport apparatus 1 (S002). The user assembles the main body 10 and the cartridge 20 to thereby cause the cam 11 on the main body 10 side to mesh with the fingers 22 on the cartridge 20 side. Additionally, the user inserts the connecting needle 233 of the cartridge 20 into the septum 33A for a connecting needle of the patch 30 and brings the connecting needles into a state where the liquid can be supplied from the cartridge 20 side to the patch 30 side.

Next, the user performs the priming processing (S003). FIG. 12 is an explanatory view of the priming processing. The priming processing is the processing of driving the pump unit 5 of the liquid transport apparatus 1 to cause the inside of the flow channel of the liquid transport apparatus 1 to be filled with the liquid. The gas within the flow channel of the liquid transport apparatus 1 is discharged from the introduction needle 32A by this priming processing. Additionally, the tube 21 in an empty state is filled of the liquid by this priming processing. The user drives the pump unit 5 of the liquid transport apparatus 1 until the liquid is discharged from the tip of the introduction needle 32A.

After the priming processing, the user perpendicularly punctures the living body with the introduction needle 32A and the soft needle 31, then pulls out the introduction needle folder 32 from the port base 33, and extracts the introduction needle 32A from the soft needle 31 (S004). Since there is the septum 33B for an introduction needle, even if the introduction needle 32A is extracted, the hole of the septum 33B for an introduction needle formed by the introduction needle 32A is spontaneously closed. At this time, the user may peel a protection sheet of the adhesion pad 35 of the patch 30 to paste the adhesion pad 35 on the skin of the living body to adhere the liquid transport apparatus 1 to the living body.

Next, the user preliminarily operates the pump unit 5 so as to transport the liquid equivalent to the volume of the region (regions other than the septum for an introduction needle) where the introduction needle 32A is present (S005). This enables a space where the introduction needle 32A is present to be filled with the liquid.

Then, the user makes the liquid transport apparatus 1 perform fixed amount transport processing (normal processing) (S006). At this time, the liquid transport apparatus 1 drives the piezoelectric motor 121 of the drive mechanism 12 to rotate the cam 11, pushes the seven fingers 22 in sequence using the projection portions of the cam 11 to make the tube 21 blocked sequentially from the upstream side in the transport direction, and causes the tube 21 to perform a peristaltic motion to transport the liquid. In the fixed amount transport processing, the rotational amount of the cam 11 is controlled so that a predetermined amount of liquid is transported in a predetermined time.

### Coming-Off Determination

If the soft needle 31 comes off the living body, the liquid is no longer injected into the living body even if the liquid transport apparatus 1 transports the liquid. In the present embodiment, since the living body is punctured by the soft needle 31 that has flexibility in order to reduce a burden on the living body, the soft needle is particularly in the state of coming off easily. Additionally, if the soft needle 31 is shortened in order to reduce the load to the living body, the soft needle 31 is brought into the state of being coming-off easily.

Thus, in the present embodiment, whether or not the soft needle 31 comes off the living body is monitored.

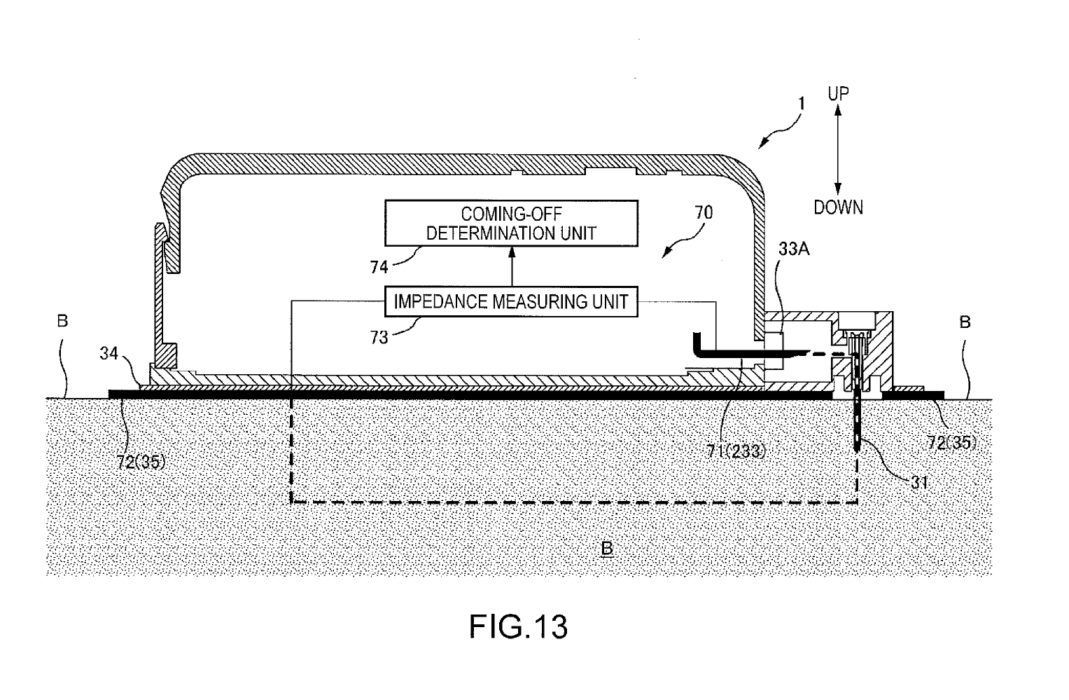

FIG. 13 is an explanatory view of a monitoring device 70 that monitors coming-off of the soft needle 31. Here, the liquid transport apparatus 1 is adhered to a living body B. The living body is punctured by the soft needle 31 in a state where the introduction needle 32A (refer to FIG. 3) is extracted. In the drawing, an electric conduction path going via the liquid and the living body B is shown by a dotted line.

The monitoring device 70 has a first electrode 71, a second electrode 72, an impedance measuring unit 73, and a coming-off determination unit 74. The impedance measuring unit 73 and the coming-off determination unit 74 are provided on the aforementioned control board 15.

The first electrode 71 is provided further toward the downstream side than a region where the fingers 22 push the tube 21. This is because, if the first electrode 71 is provided further toward the upstream side than the fingers 22, the liquid is insulated at a blocked position when the fingers 22 block the tube 21, and as a result, the impedance between the first electrode 71 and the second electrode 72 becomes high and coming-off based on the impedance cannot be determined.

The first electrode 71 is tubular and constitutes a flow channel that comes into contact with the liquid at an inner peripheral surface thereof and allows the liquid to be transported therethrough. The first electrode 71 comes into direct contact with the liquid. Therefore, compared to a case where the electrode and the liquid are capacitively coupled to each other (a case where the electrode is provided outside the tube and the electrode does not come into direct contact with the liquid), an error when the impedance is measured can be reduced, and the accuracy of the measurement improves.

Specifically, the first electrode 71 serves also as the connecting needle 233, and the connecting needle 233 is made of conductive metal. Since the connecting needle 233 is inserted through the septum 33A for a connecting needle, the connecting needle 233 is used as the first electrode 71, so that the electrode can be brought as close to the living body B side as possible.

The second electrode 72 is an electrode that is arranged so as to come into contact with the skin of the living body B. Specifically, the second electrode 72 serves also as the adhesion pad 35, and the adhesion pad 35 is constituted by a conductive pad having an adhesive face.

In addition, in order to electrically connect the impedance measuring unit 73 provided on the control board 15 on the main body 10 side and the first electrode 71 (connecting needle 233) on the cartridge 20 side, a connecting terminal (not shown) is formed between the main body 10 and the cartridge 20. Additionally, in order to electrically connect the impedance measuring unit 73 provided on the control board 15 on the main body 10 side and the second electrode 72 (adhesion pad 35) on the patch 30 side, connecting terminals (not shown) are formed between the main body 10 and the cartridge 20 and between the cartridge 20 and the patch base 34.

The impedance measuring unit 73 measures the impedance between the first electrode 71 and the second electrode 72. If the living body B is punctured by the soft needle 31 as shown, a closed circuit is configured via the liquid and the living body B. Therefore, the impedance measuring unit 73 measures the impedance of a predetermined assumed range (for example, about several 10 k) to several 100 kΩ) if an alternating voltage (for example, a frequency of about 1 kHz to 10 kHz) is applied to the first electrode 71 and the second electrode 72. On the other hand, if the soft needle 31 comes off the living body B, the closed circuit is not configured and an electric current does not flow. Therefore, the impedance measuring unit 73 measures a high impedance (for example, equal to or higher than 10 MΩ).

The coming-off determination unit 74 determines the coming-off of the soft needle 31 on the basis of a measurement result of the impedance measuring unit 73. Specifically, the coming-off determination unit 74 determines that the living body B is normally punctured by the soft needle 31 if the impedance that is the measurement result is equal to or lower than a predetermined threshold. Additionally, the coming-off determination unit 74 determines that the soft needle 31 comes off the living body B if the impedance that is the measurement result is higher than the predetermined threshold.

The coming-off determination unit 74 outputs the measurement result to the control unit of the control board 15. The control unit continues the fixed amount transport processing (S006) when it is determined that the living body B is normally punctured by the soft needle 31, and stops the fixed amount transport processing and notifies the user of warning with sound, light, or the like when it is determined that the soft needle 31 comes off the living body B.

As described above, the liquid transport apparatus 1 of the present embodiment includes the tube 21 for transporting the liquid, the plural fingers 22 that push and block the tube, the cam 11 that pushes the fingers in sequence so that the tube is squeezed to transport the liquid, and the soft needle 31 serving as a catheter. In order to determine that the soft needle 31 comes off the living body B, the liquid transport apparatus 1 includes the first electrode 71, the second electrode 72, and the coming-off determination unit 74, and performing coming-off determination based on the impedance using the fact that the impedance becomes high if the soft needle 31 comes off the living body B.

Additionally, the inner peripheral surface of the first electrode 71 constitutes a flow channel that comes into contact with the liquid and allows the liquid to be transported therethrough. By bringing the first electrode 71 into contact with the liquid in this way, an error when the impedance is measured can be reduced, and the accuracy of the measurement improves, compared to a case where the first electrode 71 and the liquid are capacitively coupled to each other (a case where the first electrode 71 is provided outside the tube and the first electrode 71 does not come into direct contact with the liquid).

In addition, the first electrode 71 is provided further toward the downstream side than a region where the fingers 22 push the tube 21. The reason is because, if the first electrode 71 is provided further toward the upstream side than the fingers 22, the liquid is insulated at a blocked position when the fingers 22 block the tube 21, and coming-off based on the impedance cannot be determined.

Additionally, in the present embodiment, the cartridge 20 has the connecting needle 233 serving as the first electrode 71, and the patch 30 has the soft needle 31 serving as a catheter, the adhesion pad 35 serving as the second electrode 72, and the septum 33A for a connecting needle. Since the connecting needle 233 is inserted through the septum 33A for a connecting needle, the connecting needle 233 is used as the first electrode 71, so that the electrode can be brought as close to the living body B side as possible.

Additionally, in the present embodiment, the soft needle 31 is made of an insulating material. This can prevent the liquid passing through the soft needle 31 and the second electrode 72 (adhesion pad 35) from short-circuiting.

Additionally, in the present embodiment, the second electrode 72 is constituted by a conductive adhesion pad. This can increase the area by which the second electrode 72 comes into contact with the living body.

Additionally, in the present embodiment, the adhesion pad 35 serving as the second electrode 72 is formed with a hole for allowing the soft needle 31 serving as a catheter to pass therethrough. Since this allows the adhesion pad 35 to be arranged around the catheter, the soft needle 31 does not come off easily. Additionally, since the injection port of the soft needle 31 is arranged in the vicinity of the second electrode 72, the conduction path going via the liquid and the living body B is easily formed.

In addition, in the present embodiment, the whole surface of the adhesion pad 35 is made to have conductivity. However, a portion of the adhesion pads 35 may be made to have conductivity, and the portion having conductivity may be used as the second electrode 72. In addition, it is preferable to make only a central portion of the adhesion pad 35 have conductivity (making a peripheral portion of the adhesion pad 35 not have conductivity) so as to use the central portion of the adhesion pad 35 as the second electrode 72. Accordingly, there is an effect that measurement of the impedance is not easily influenced even if the peripheral portion having conductivity is peeled off.

Additionally, in the present embodiment, an alternating voltage is applied in a state where a DC component of a supply voltage of the impedance measuring unit 73 is cut so that a bias voltage is not applied between the first electrode 71 and the second electrode 72. This is because, if a DC voltage is applied between the first electrode 71 and the second electrode 72, an electrochemical process occurs in the liquid (liquid between the first electrode 71 and the second electrode 72) that comes in contact with the electrodes, a possibility that the characteristics of the liquid may change or precipitate may adhere to the electrodes occurs.

### Others

The embodiment is merely for facilitating the understanding of the invention and is not for limitedly interpreting the invention. It is apparent that the invention may be modified and improved without departing from the spirit thereof and the equivalents thereof are included in the invention.

### Monitoring Device 70

In the aforementioned monitoring device 70, the impedance measuring unit 73 measures the value of the impedance between the first electrode 71 and the second electrode 72 with high precision, and the coming-off determination unit 74 determines the coming-off of the catheter (soft needle 31) on the basis of the value of the impedance. However, it is also acceptable if the value of the impedance is not measured with high precision.

For example, the impedance measuring unit may be configured so that an L-level (or H-level) signal is output when the impedance between the first electrode 71 and the second electrode 72 is higher than a predetermined value, and an H-level (or L-level) signal is output when the impedance is equal to or lower than the predetermined value, and the coming-off determination unit 74 may determine the coming-off of the catheter on the basis of the output signal of the impedance measuring unit.

### Electrode

Although the first electrode of the aforementioned embodiment serves also as the connecting needle 332 and the second electrode serves also as the adhesion pad 35, the first electrode and the second electrode are not intended to be limited to this.

For example, the first electrode may be used as the discharge-side joint 232. In this case, however, since the first electrode is arranged further apart from the living body than the aforementioned embodiment, an error is easily caused in measurement of the impedance.

Additionally, the first electrode and the second electrode may be independently provided without being made to serve also as other constituent elements. However, the number of parts will increase more than the aforementioned embodiment.

The entire disclosure of Japanese Patent Application No. 2012-238673, filed Oct. 30, 2012 is expressly incorporated by reference herein.

What is claimed is:

1. A liquid transport apparatus comprising:
a catheter for transporting a liquid to a living body;
a first electrode that comes into contact with the liquid;
a second electrode that is arranged at the catheter; and
a determination unit that determines that the catheter comes off the living body on the basis of the impedance between the first electrode and the second electrode.

2. The liquid transport apparatus according to claim 1, further comprising:
a cartridge and a patch,
wherein the cartridge has a connecting needle serving as the first electrode,
the patch has the catheter, the second electrode, and a septum, and
the liquid of the cartridge is transported to the catheter via the connecting needle by the connecting needle being inserted through the septum.

3. The liquid transport apparatus according to claim 1,
wherein the catheter is made of an insulating material.

4. The liquid transport apparatus according to claim 1,
wherein the second electrode is constituted by a conductive adhesion pad.

5. The liquid transport apparatus according to claim 4,
wherein the adhesion pad is formed with a hole through which the catheter passes.

6. The liquid transport apparatus according to claim 4,
wherein a peripheral portion of the adhesion pad has no conductivity, and a central portion of the adhesion pad has conductivity.

7. The liquid transport apparatus according to claim 1,
wherein an alternating voltage is applied to the first electrode and the second electrode when the impedance between the first electrode and the second electrode is measured.

8. A catheter coming-off determination method of a liquid transport apparatus including a catheter for transporting a liquid to a living body, a first electrode, and a second electrode, the method comprising:
determining that the catheter comes off the living body on the basis of the impedance between the first electrode that comes into contact with the liquid and the second electrode that is arranged at the catheter.
